(19) United States
(12) Patent Application Publication
(10) Pub. No.: US 2005/0080065 A1
(43) Pub. Date: Apr. 14, 2005

(54) Process for the production of 6.alpha.,9.alpha-difluoro-17.alpha.-(1-oxo-propoxy-11.beta.-hydroxy-16.alpha.-methyl-3-oxo-androst-1,4-diene-17.beta.-carbothioic acid

(76) Inventors: Steven John Coote, Stevenage (GB); Rosalyn Kay Nice, Stevenage (GB); Mark David Wipperman, Stevenage (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/502,684

(22) PCT Filed: Feb. 3, 2003
(86) PCT No.: PCT/EP03/01116

(30) Foreign Application Priority Data
Feb. 4, 2002 (GB) 0202563.3

(51) Int. Cl. A61K 31/56; C07J 7/00

(52) U.S. Cl. 514/179; 552/580

(57) ABSTRACT

A process for preparing a compound of formula (II) 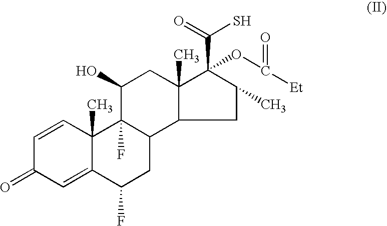 or a salt thereof is described.

This invention relates to a novel process for preparing a chemical intermediate useful in the preparation of fluticasone propionate.

Fluticasone propionate is a corticosteroid of the androstane family which has potent anti-inflammatory activity and is widely accepted as a useful therapy for the treatment of inflammatory and allergic conditions such as rhinitis and asthma. The chemical representation of fluticasone propionate is as shown in the structure below:

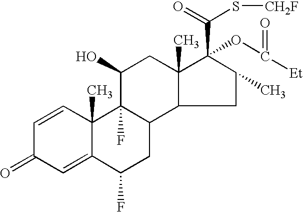

One process for preparing fluticasone propionate comprises reacting a compound of formula (II) 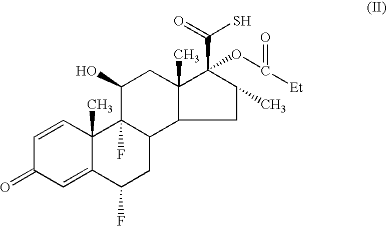
with a compound of formula LCH₂F, where L represents a leaving group mesyl, tosyl or halogen, eg Cl, Br or I. Preferably L represents halogen, particularly Br.

According to prior art processes, eg as described in G. H. Phillips et al (1994) J Med Chem 37, 3717-3729 and U.S. Pat. No. 4,335,121 (Glaxo Group Limited), compounds of formula (II) may be prepared by reacting a compound of formula (III) 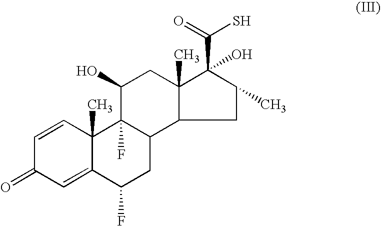
with an activated derivative of propionic acid eg propionyl chloride. The activated derivative of propionic acid will generally be employed in at least 2 times molar quantity relative to the compound of formula (III) since one mole of the reagent will react with the thioacid moiety and needs to be removed eg by reaction with an amine such as diethylamine.

This method for the preparation of compound of formula (II) suffers from disadvantages, however, in that the resultant compound of formula (II) is not readily purified of contamination with the by-product N,N-diethylpropanamide. We have therefore invented an improved process for performing the conversion to prepare compound of formula (II).

Other processes for preparation of fluticasone propionate and related compounds are described in Israeli patent aplication 109656 (Chemagis), WO01/62722 (Abboft) and Kertesz and Marx (1986) J Org Chem 51, 2315-2328.

Thus according to the invention there is provided a process for preparing a compound of formula (II) 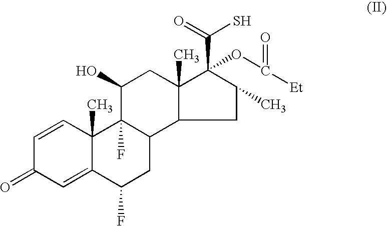
or a salt thereof
which comprises:
(a) reacting a compound of formula (III) 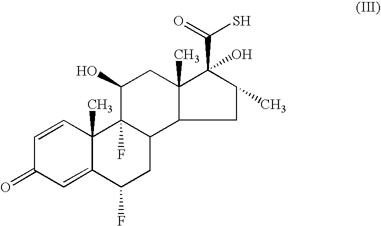
with an activated derivative of propionic acid in an amount of at least 1.3 moles, suitably at least 2 moles, of the activated derivative per mole of compound of formula (III) and;
(b) removal of the sulphur-linked propionyl moiety from any compound of formula (IIA) 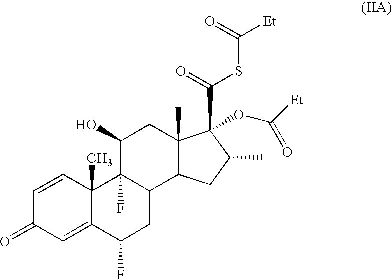
so formed by reaction of the product of step (a) with an organic primary or secondary amine base capable of forming a water soluble propanamide.

In step (a), examples of activated derivatives of propionic acid include activated esters or preferably a propionyl halide such as propionyl chloride. This reaction is conventionally performed in the presence of an unreactive organic base such as a triC₁₋₄alkylamine eg tri-n-propylamine, triethylamine, or tributylamine especially triethylamine, but most preferably tri-n-propylamine. Solvents for this process include substantially water immiscible solvents such as ethyl acetate or methyl acetate or water miscible solvents such as acetone, N,N-dimethylformamide or N,N-dimethylacetamide, especially acetone. Substantially immiscible solvents provide two phases when the solvents are mixed and have a low level of solubility one in the other eg the solubility of one solvent in the other solvent is less than 30% w/w, for example 10% w/w, especially 5% w/w.

In step (b), examples of organic primary or secondary amine base capable of forming a water soluble propanamide include amines which are more polar than diethylamine, for example an alcoholamine, e.g. diethanolamine, or a diamine, for example N-methylpiperazine. Preferably, N-methylpiperazine is employed. It may be convenient to dissolve the amine in small volume of an organic solvent such as methanol.

Preferably steps (a) and (b) are performed at reduced temperature eg 0-5° C.

As a further aspect of the invention we provide methods for the efficient purification of the compound of formula (II).

Thus a first such process (c1) comprises, when the product of step (b) is dissolved in a substantially immiscible organic solvent (such as methyl acetate or ethyl acetate, or a higher alkanone such as a pentan-3-one), purifying the compound of formula (II) by washing out the amide by-product from step (b) with an aqueous wash. For example water may be added to the reaction mixture, stirred, the phases allowed to separate, and the lower aqueous layer run off.

Preferably, the substantially immiscible solvent is pentan-3-one.

The remaining organic layer may be concentrated by distillation optionally at reduced pressure and then an antisolvent (eg hexane) may be added to crystallise the dissolved product.

A second such process (c2) comprises, when the product of step (b) is dissolved in a water miscible solvent (eg acetone), purifying the compound of formula (II) by treating the compound of step (b) with an aqueous medium so as to precipitate out pure compound of formula (II). The amide by-product from step (b) will accordingly substantially remain in the aqueous phase.

The aqueous medium may, for example, be a dilute aqueous acid such as dilute hydrochloric acid or acetic acid.

Further general conditions pertaining to the conversion of the compound of formula (III) to the compound of formula (II) and salts thereof and the isolation of the end product will be well known to persons skilled in the art.

According to a preferred set of conditions, however, we have found that the compound of formula (II) may advantageously be isolated following process (c1) in the form of a solid crystalline salt rather than the free compound of formula (II). The preferred salt is formed with a base such as diisopropylethylamine, triethylamine, 2,6-dimethylpyridine, N-ethylpiperidine or with potassium. Such salt forms of compound of formula (II) are more stable, more readily filtered and dried and can be isolated in higher purity than the free compound of formula (II). The most preferred salt is the salt formed with triethylamine. The potassium salt is also of interest.

Thus a preferred process, following process (c1), comprises treating the organic phase containing the compound of formula (II) with a base so as to precipitate the compound of formula (II) in the form of a solid crystalline salt.

Example bases include triethylamine, 2,6-dimethylpyridine, N-ethylpiperidine or a basic potassium salt eg potassium hydrogen carbonate.

We claim a compound of formula (II) isolated in the form of a solid crystalline salt as a further aspect of the invention.

As a further aspect of the invention we also provide a process for preparing fluticasone propionate which comprises preparing a compound of formula (II) or a salt thereof as just described and converting the compound of formula (II) or salt thereof to fluticasone propionate by treatment with a compound of formula LCH₂F wherein L represents a leaving group.

According to the invention compound of formula (II) may advantageously be isolated with higher efficiency than by means of prior art processes. For example, the process for the preparation of the compound of formula (II) disclosed in G. H. Phillips et al (1994) J Med Chem 37, 3717-3729 involves the isolation of the product from an acetone/water system. The product so prepared is extremely difficult to filter. In contrast, the compound of formula (II), when prepared in accordance with the present invention, is far easier to filter. Furthermore, the process of the present invention may also offer improvements in purity.

It is additionally advantageous to prepare and use the compound of formula (III) as an imidazole salt. G. H. Phillips et al (1994) J Med Chem 37, 3717-3729 disclose the preparation of a compound of formula (III) from a compound of formula (IV). However, the physicochemical properties of the compound of formula (III) so prepared result in a product which has a very low speed of filtration. The advantages conferred by the preparation of the imidazole salt of the compound of formula (III) include its properties as an easily prepared and rapidly-filterable, easily handled and stored source of a compound of formula (III), which compound may readily be obtained from the salt by acidification, for example with hydrochloric acid. Furthermore, the compound of formula (III) so derived is of enhanced purity. The imidazole salt of the compound of formula (III) may be prepared, isolated, and stored for subsequent use in the process for the preparation of the compound of formula (II) as described herein. Alternatively, the imidazole salt of the compound of formula (III) may be prepared and used directly as a wet cake in the subsequent conversion to a compound of formula (II) thus avoiding the need to dry the imidazole salt before further reaction.

It is considered that the imidazole salt of the compound of formula (III) is new and accordingly forms a further aspect of the invention. There is also further provided a process for the preparation of the imidazole salt of the compound of formula (III) which process comprises the reaction of a compound of formula (IV) with carbonyldiimidazole and hydrogen sulphide.

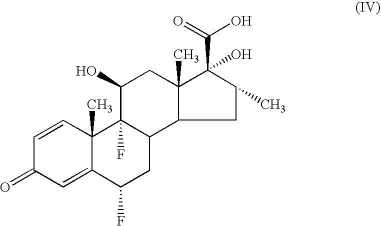

Typically, the compound of formula (IV) and between 1.1 and 2.5 equivalents, suitable 1.8 equivalents, of carbonyldiimidazole are stirred in a suitable solvent, for example ethyl acetate containing between 0 and 2 vol., suitably 0.5 vol., of N,N-dimethylformamide, at a suitable temperature, for example 18-20° C., for a suitable period of time, for example one hour. The resulting suspension is cooled to a suitable temperature, for example −5 to 5° C., suitably −3 to 3° C., and hydrogen sulphide gas introduced over a period of 15-60 minutes, suitably 20-30 minutes, while the suspension is stirred. The reaction mixture is stirred for a further period of about 30 minutes at −5 to 5° C., warmed to about 10° C. over a period of about 20 minutes and stirred at 6-12° C. for 90-120 minutes. The product is then isolated by filtration, at a suitable temperature, suitably 5-25° C., preferably 10-15° C., washed with a suitable solvent, for example ethyl acetate, and dried in vacuo to yield the imidazole salt of the compound of formula (III).

The compound of formula (III) is a monobasic acid and therefore would be expected to form an imidazole salt wherein the stoichiometry of the imidazole salting moiety to the compound of formula (III) is approximately 1:1. However, it has surprisingly been found that the stoichiometry of the imidazole salting moiety to the compound of formula (III) may be up to and including 4:1. Therefore, for the avoidance of doubt, the term “imidazole salt” encompasses imidazole salts of the compound of formula (III) and association compounds of the compound of formula (III) and imidazole wherein the stoichiometry of the imidazole moiety to the compound of formula (III) is up to and including 4:1, for example 1:1 to 4:1, suitably 1.8:1 to 2.5:1. An example of a typical stoichiometry is 2:1. It will be understood that, in the context of stoichiometric values, exact numerical values are to be construed to include nominal variations therefrom.

Preferably, the compound of formula (III) used in the process described herein is used as its imidazole salt.

The invention will be illustrated with reference to the following examples.

### EXAMPLES

General

¹H-nmr spectra were recorded at 400 MHz and the chemical shifts are expressed in ppm relative to tetramethylsilane. The following abbreviations are used to describe the multiplicities of the signals: s (singlet), d (doublet), t (triplet), q (quartet), m (multiplet), dd (doublet of doublets), ddd (doublet of doublet of doublets), dt (doublet of triplets) and b (broad).

LCMS was conducted on a 25 cm×0.46 cm Inertsil ODS-2, 5 μm column eluting with 58% {0.1% Formic acid in 3% methanol (aqueous)} (solvent A), and 42% {0.1% Formic acid in 3% methanol (acetonitrile)} (solvent B), using the 1.0 following elution gradient 0-40 min 42% B, 40-60 min 53% B, 60-75 min 87% B, 75-85 min 42% B at a flow rate of 1 ml/min. The mass spectra were recorded on a HP LC/MSD spectrometer using electrospray positive and negative mode (ES+ve and ES−ve).

Liquid Chromatography (Method A) was conducted on a 25 cm×0.46 cm ID packed with 5 μm Inertsil ODS-2 column eluting with the following acidified mobile phases:
Solution A: Acidified Acetonitdle: Acidified Methanol: Acidified Water (42:3:55)
Solution B: Acidified Acetonitrile: Acidified Methanol: Acidified Water (53:3:44)
Solution C: Acidified Acetonitrile: Acidified Methanol: Acidified Water (87:3:10)
   {where acidified acetonitrile comprises of 0.05% v/v Phosphoric acid in acetonitrile (0.5 ml in 1000 ml), acidified methanol comprises of 0.05% v/v Phosphoric acid in methanol (0.5 ml in 1000 ml) and acidified water comprises of 0.05% v/v Phosphoric acid in water (0.5 ml in 1000 ml)}.
      The following elution gradient 0-40 min Solution A (100%), 40-60 min Solution B (100%), 60-75 min Solution C (100%) and 75-90 min Solution A (100%) was run at a flow rate of 1.0 ml/minute at oven temperature of 40° C.

Liquid Chromatography (Method B) was conducted on a Stainless steel 5 μm Octyl 20 cm×0.46 cm id column eluting with the following acidified mobile phases:
Solution A: Acetonitrile: 0.05M aqueous ammonium dihydrogen orthophosphate (35:65) by volume
Solution B: Acetonitrile: 0.05M aqueous ammonium dihydrogen orthophosphate (70:30) by volume
   The following elution gradient 0-15 min Solution A (100%), 15-40 min Solution B (100%), 40-45 min Solution B (100%) and 45-60 min Solution A (100%) was run at a flow rate of 1.5 ml/minute at oven temperature of 30° C.

Melting points were obtained using Mettler Toledo FP62 melting point apparatus.

XRPD's were obtained using a Phillips X'pert MPD powder diffractometer.

### Example 1

### 6α,9α-Difluoro-17α-(1-oxopropoxy)-11β-hydroxy-16α-methyl-3-oxo-androsta-1,4-diene-17β-carbothioic Acid (Using N,N-dimethylformamide as the Water Miscible Solvent)

A solution of 6α,9α-difluoro-11β,17α-dihydroxy-16α-methyl-3-oxo-androsta-1,4-diene-17β-carbothioic acid (prepared in accordance with the procedure described in GB 2088877B) (7 g) in N,N-dimethylformamide (45 ml) is treated at −5° C. to −6° C. with triethylamine (10.9 ml) over approximately 15 minutes. The solution is stirred at −5° C. to 0° C. during the addition followed by a wash with N,N-dimethylformamide (2.8 ml). The resultant suspension is further cooled to −3° C. to −7° C. and treated with propionyl chloride (6.2 ml) over approximately 30 minutes, maintaining the temperature at −5° C. to +2° C. N,N-Dimethylformamide (2.8 ml) is added as a line wash. The solution is stirred at −5° C. to +2° C. for a further 2 hours. The resultant suspension is further cooled to −3° C. to −7° C. and treated with diethanolamine (23.8 ml) in methanol (20 ml) over approximately 30 minutes, maintaining the temperature at −5° C. to +2° C. N,N-Dimethylformamide (2.8 ml) is added as a line wash and the solution is stirred at −5° C. to +2° C. for a further 30 minutes. Chilled hydrochloric acid (comprising a mixture of 20 ml concentrated HCl and 20 ml water) is added maintaining the temperature in the range of −5° C. to +5° C. over approximately 30 minutes and the mixture is quenched into cooled dilute hydrochloric acid (comprising a mixture of 50 ml concentrated hydrochloric acid and 300 ml water) over approximately 30 minutes, maintaining the temperature in the range of −5° C. to +5° C. Aqueous N,N-dimethylformamide (comprising a mixture of 10 ml N,N-dimethylformamide and 20 ml water) is added as a vessel wash and the resultant suspension is aged at −5° C. to +5° C. for at least 10 minutes. The product is filtered off, washed with water and dried under vacuum at approximately 45° C. for 24 hours to give the title compound as a white to off white solid (6.65 g, 83.7%).

HPLC retention time 27.23 min, m/z 469.2 (positive molecular ion) and m/z 467.2 (negative molecular ion).

NMR (DMSO_{d6}) 7.27 (1H, d, 10 Hz, 6.34 (1H, d, 10 Hz), 6.14 (1H, s), 5.31 (1H, d), 5.17 (1H, ddd), 4.27 (1H, m), 2.40 (2H, q, 7 Hz), 2.00-2.14 (5H, m), 1.85 (1H, m), 1.65 (1H, m), 1.51 (3H, s), 1.14 (3H, s), 1.05 (3H, t, 7 Hz), 0.88 (3H, d, 7 Hz).

### Example 2

### 6α,9α-Difluoro-17α-(1-oxopropoxy)-11β-hydroxy-16α-methyl-3-oxo-androsta-1,4-diene-17β-carbothioic Acid (Using Acetone as the Water Miscible Solvent)

A solution of 6α,9α-difluoro-11β,17α-dihydroxy-16α-methyl-3-oxo-androsta-1,4-diene-17β-carbothioic acid (prepared in accordance with the procedure described in GB 2088877B) (7 g) in acetone (80.6 ml) is treated at −5° C. to −6° C. with triethylamine (10.9 ml) over approximately 15 minutes. The solution is stirred at −5° C. to 0° C. during the addition followed by a wash with acetone (2.8 ml). The resultant suspension is further cooled to −3° C. to −7° C. and treated with propionyl chloride (6.2 ml) over approximately 30 minutes, maintaining the reaction temperature at −5° C. to +2° C. Acetone (2.8 ml) is added as a line wash and the solution is stirred at −5° C. to +2° C. for a further 2 hours. The resultant suspension is further cooled to −3° C. to −7° C. and treated with diethanolamine (23.8 ml) in methanol (20 ml) over approximately 30 minutes, maintaining the temperature at −5° C. to +2° C. Acetone (2.8 ml) is added as a line wash and the solution is stirred at −5° C. to +2° C. for a further 30 minutes. The mixture is quenched into water (135 ml) maintaining the temperature at −5° C. to +5° C. Acetone (5.6 ml) is added as a line wash and the mixture is cooled to 0° C. to 5° C. Concentrated hydrochloric acid (65 ml) is added over one to two hours maintaining the temperature in the range of 0° C. to 5° C. followed by addition of water (125 ml) maintaining the temperature at <5° C. The mixture is stirred at 0° C. to 5° C. for 15 minutes, the product is filtered off, washed with water and dried under vacuum at approximately 45° C. for 18 hours to give the title compound as a white to off white solid (7.91 g, 99.5%).

### Example 3

### 6α,9α-Difluoro-17α-(1-oxopropoxy)-11β-hydroxy-16α-methyl-3-oxo-androsta-1,4-diene-17β-carbothioic Acid (Using N,N-dimethylacetamide as the Water Miscible Solvent)

A solution of 6α,9α-difluoro-11β,17α-dihydroxy-16α-methyl-3-oxo-androsta-1,4-diene-17β-carbothioic acid (prepared in accordance with the procedure described in GB 2088877B) (7 g) in N,N-dimethylacetamide (40 ml) is treated at −5° C. to 0° C. with triethylamine (10.9 ml) over approximately 15 minutes, followed by a line wash with N,N-dimethylacetamide (2.8 ml). The resultant suspension is further cooled to −3° C. to −7° C. and treated with propionyl chloride (6.2 ml) over approximately 30 minutes, maintaining the temperature at −5° C. to +2° C. N,N-Dimethylacetamide (2.8 ml) is added as a line wash and the solution is stirred at −5° C. to +2° C. for a further 2 hours. The resultant suspension is further cooled to −3° C. to −7° C. and treated with diethanolamine (23.8 ml) in methanol (20 ml) over approximately 30 minutes, maintaining the temperature at −5° C. to +2° C. N,N-Dimethylacetamide (2.8 ml) is added as a line wash and the solution is stirred at −5° C. to +2° C. for a further 30 minutes. Chilled hydrochloric acidic (comprising a mixture of 10 ml concentrated hydrochloric acid and 30 ml water) is added maintaining the temperature in the range of −5° C. to +5° C. over approximately 30 minutes. The reaction mixture is quenched into dilute hydrochloric (comprising a mixture of 55 ml concentrated hydrochloric acid and 300 ml water) over approximately 30 minutes maintaining the temperature at −5° C. to +5° C. during the transfer. Aqueous N,N-dimethylacetamide (comprising a mixture of 10 ml N,N-dimethylformamide and 20 ml water) is added as a line wash and the resultant suspension is aged at −5° C. to +5° C. for at least 10 minutes. The product is filtered off, washed with water and dried under vacuum at approximately 45° C. for 24 hours to give the title compound as a white to off white solid (7.63 g, 96%).

### Example 4

### 6α,9α-Difluoro-17α-(1-oxopropoxy)-11β-hydroxy-16α-methyl-3-oxo-androsta-1,4-diene-17β-carbothioic Acid (Using Ethyl Acetate as the Substantially Water Immiscible Solvent)

A solution of 6α,9α-difluoro-11β,17α-dihydroxy-16α-methyl-3-oxo-androsta-1,4-diene-17β-carbothioic acid (prepared in accordance with the procedure described in GB 2088877B) (7 g) in ethyl acetate (350 ml) is stirred at 20 to 25° C. and treated at 0 to 5° C. with triethylamine (10.9 ml) over approximately 20 minutes adding further ethyl acetate (5 ml) as a line wash. The resultant suspension is further cooled to −3° C. to −7° C. and treated with propionyl chloride (6.2 ml) over approximately 30 minutes, maintaining the temperature at −5° C. to +2° C. Ethyl acetate (5 ml) is added as a line wash and the solution is stirred at −5° C. to +2° C. for a further 2 hours. The resultant suspension is further cooled to −3° C. to −7° C. and treated with diethanolamine (23.8 ml) in methanol (20 ml) over approximately 30 minutes, maintaining the temperature at −5° C. to +2° C. Ethyl acetate (5 ml) is added as a line wash and the solution is stirred at −5° C. to +2° C. for a further 30 minutes. Acetic acid (25 ml) is added maintaining the temperature in the range of −5° C. to +2° C. over approximately 10 minutes and the resultant suspension is aged at −5° C. to +5° C. for at least 10 minutes. Water (50 ml) is added over approximately 10 minutes maintaining the temperature in the range of −5° C. to +2° C. and the organic phase is separated and washed with water (3×50 ml). The aqueous phases are optionally back extracted with ethyl acetate (120 ml) at −5° C. to +2° C. The combined organic phases are concentrated to 10 vol. by vacuum distillation (below 30° C.) and cooled to 0° C. to 5° C. Hexane is added (70 ml) maintaining the temperature at 0° C. to 5° C. and the contents aged for at least 30 minutes at 0° C. to 5° C. The product is filtered off and washed twice with a cooled mixture (0° C. to 5° C.) of ethyl acetate (49 ml) and hexane (49 ml). The product is dried under vacuum at approximately 45° C. for 18 hours to give the title compound as a white to off white solid (6.37 g, 80.6%).

### Example 5

### 6α,9α-Difluoro-17α-(1-oxopropoxy)-11β-hydroxy-16α-methyl-3-oxo-androsta-1,4-diene-17β-carbothioic Acid S-Fluoromethyl Ester (Using a Single Solvent to Avoid Isolation of Intermediate 6α,9α-difluoro-17α-(1-oxopropoxy)-11β-hydroxy-16α-methyl-3-oxo-androsta-1,4-diene-17β-carbothioic Acid)

A solution of 6α,9α-difluoro-11β,17α-dihydroxy-16α-methyl-3-oxo-androsta-1,4-diene-17β-carbothioic acid (prepared in accordance with the procedure described in GB 2088877B) (7 g) in ethyl acetate (350 ml) is stirred at 20 to 25° C. and treated at 0 to 5° C. with triethylamine (10.9 ml) over approximately 20 minutes adding further ethyl acetate (5 ml) as a line wash. The resultant suspension is further cooled to −3° C. to −7° C. and treated with propionyl chloride (6.2 ml) over approximately 30 minutes, maintaining the temperature at −5° C. to +2° C. Ethyl acetate (5 ml) is added as a line wash and the solution is stirred at −5° C. to +2° C. for a further 2 hours. The resultant suspension is further cooled to −3° C. to −7° C. and treated with diethanolamine (23.8 ml) in methanol (20 ml) over approximately 30 minutes, maintaining the temperature at −5° C. to +2° C. Ethyl acetate (5 ml) is added as a line wash and the solution is stirred at −5° C. to +2° C. for a further 30 minutes. Acetic acid (25 ml) is added maintaining the temperature in the range of −5° C. to +2° C. over approximately 10 minutes and the resultant suspension is aged at −5° C. to +5° C. for at least 10 minutes. Water (30 ml) is added over approximately 10 minutes maintaining the temperature in the range of −5° C. to +2° C. and the organic phase is separated and washed with water (3×50 ml). The aqueous phases are optionally back extracted with ethyl acetate (120 ml) at −5° C. to +2° C. and the combined organic phases are concentrated to approximately 45 vol. by vacuum distillation (below 10° C.). Approximately one half of the resultant solution is treated with water (13.5 ml), benzyltributylammonium chloride (0.37 g) and triethylamine (1.3 ml) and the mixture is cooled to 5° C. Bromofluoromethane (0.5 ml) is added maintaining a reaction temperature of approximately 5° C. The mixture is warmed to 20° C. over 2-3 hrs and the resultant suspension is sequentially washed with 0.5M hydrochloric acid (23 ml), 1% w/w aqueous sodium bicarbonate solution (3×23 ml) and water (2×23 ml). The organic layer is separated and the aqueous layer is back extracted with ethyl acetate (30 ml). The combined organic layers are distilled to an approximate volume 22 ml and further ethyl acetate (7 ml) is added. The mixture is cooled to approximately 20° C., hexane (42 ml) is added over at least 30 minutes and the mixture is aged at 20° C. for 15 minutes. The resultant precipitate is collected by filtration, washed with 1:4 ethyl acetatethexane (3×5 ml) and dried at approximately 50° C. for 18 hours to give the title compound as a white solid (3.54 g, 95.7%).

The process was successfully repeated employing pentan-3-one as solvent in place of ethyl acetate.

### Example 6

### 6α,9α-Difluoro-17α-(1-oxopropoxy)-11β-hydroxy-16α-methyl-3-oxo-androsta-1,4-diene-17β-carbothioic Acid Triethylamine Salt (Using Ethyl Acetate as the Substantially Water Immiscible Solvent)

A solution of 6α,9α-difluoro-11β,17α-dihydroxy-16α-methyl-3-oxo-androsta-1,4-diene-17β-carbothioic acid (prepared in accordance with the procedure described in GB 2088877B) (7 g) in ethyl acetate (350 ml) is stirred at 20 to 25° C. and treated at 0 to 5° C. with triethylamine (10.9 ml) over approximately 20 minutes adding further ethyl acetate (5 ml) as a line wash. The resultant suspension is further cooled to −3° C. to −7° C. and treated with propionyl chloride (6.2 ml) over approximately 30 minutes, maintaining the temperature at −5° C. to +2° C. Ethyl acetate (5 ml) is added as a line wash and the solution is stirred at −5° C. to +2° C. for a further 2 hours. The resultant suspension is further cooled to −3° C. to −7° C. and treated with diethanolamine (23.8 ml) in methanol (20 ml) over approximately 30 minutes, maintaining the temperature at −5° C. to +2° C. Ethyl acetate (5 ml) is added as a line wash and the solution is stirred at −5° C. to +2° C. for a further 30 minutes. Acetic acid (25 ml) is added maintaining the temperature in the range of −5° C. to +2° C. over approximately 10 minutes and the resultant suspension is aged at −5° C. to +5° C. for at least 10 minutes. Water (50 ml) is added over approximately 10 minutes maintaining the temperature in the range of −5° C. to +2° C. and the organic phase is separated and washed with water (3×50 ml). The aqueous phases are optionally back extracted with ethyl acetate (120 ml) at −5° C. to +2° C. The combined organic phases are treated with triethylamine (15 ml), followed by azeotropic distillation (with ethyl acetate) until the batch turns cloudy. The content of the batch is then adjusted to 5 vol. by distillation or by top-up with ethyl acetate if the volume is below 5 vol. The resultant solution is then cooled to 0° C. to 5° C. Hexane is added (70 ml) maintaining the temperature at 0° C. to 5° C. and the contents aged for at least 30 minutes at 0° C. to 5° C. The product is filtered off and washed twice with a cooled mixture (0° C. to 5° C.) of ethyl acetate (49 ml) and hexane (49 ml). The product is dried under vacuum at approximately 45° C. for 18 hours to give the title compound as a white to off white solid (7.75 g, 80.6%).

### Example 7

### 6α,9α-Difluoro-17α-(1-oxopropoxy)-11β-hydroxy-16α-methyl-3-oxo-androsta-1,4-diene-17β-carbothioic Acid Triethylamine Salt (Alternative Process)

A solution of 6α,9α-difluoro-17α-(1-oxopropoxy)-11β-hydroxy-16α-methyl-3-oxo-androsta-1,4-diene-17β-carbothioic acid (prepared in accordance with the procedure described in example 2) (15 g) in acetone (90 ml) is cooled to approximately 15° C. and treated with triethylamine (5 ml) in acetone (20 ml). The reaction mixture is aged at approximately 20° C. for 0.5 hr before cooling to 15° C. Hexane (50 ml) is added over 15 minutes and the mixture is aged for 0.5 hrs. The product is filtered off, washed with chilled hexane (120 ml) and ethyl acetate (30 ml) and dried under vacuum at approximately 22° C. to give the title compound as a white to off white solid (17.72 g, 94% th).

MPt 164.3° C.

¹H NMR 5 (CD₃OD) 7.21 (1H, d), 6.44 (1H, s,), 6.39 (1H, d), 5.46 (1H, ddd), 4.37 (1H, m), 3.69 (1H, bs), 3.21 (6H, m), 2.15-2.55 (7H, m), 1.54 (3H, s), 1.49 (1H, s), 1.38 (9H, m), 1.15-1.25 (1H, m), 1.10-1.15(6H, m), 0.97 (3H, d).

In a similar manner the following salts of 6α,9α-difluoro-17α-(1-oxopropoxy)-11β-hydroxy-16α-methyl-3-oxo-androsta-1,4-diene-17β-carbothioic acid were prepared by substituting the triethylamine used in Example 7 with the relevant base. All salts were confirmed to be of high purity by HPLC:

### 6α,9α-Difluoro-17α-(1-oxopropoxy)-11β-hydroxy-16α-methyl-3-oxo-androsta-1,4-diene-17β-carbothioic Acid Potassium Salt

A solution of 6α,9α-difluoro-17α-(1-oxopropoxy)-11β-hydroxy-16α-methyl-3-oxo-androsta-1,4-diene-17β-carbothioic acid (2 g) in acetone (51 ml) and water (5 ml) is treated with potassium bicarbonate (0.46 g) and the mixture is stirred at ambient temperature until dissolution is obtained. The reaction mixture is concentrated using vacuum distillation to around 8 ml before layering with hexane and dichloromethane until white solids started to precipitate out. The mixture is cooled to 15° C. overnight. The product is filtered off, washed with hexane (16 ml) and ethyl acetate (4 ml) and dried under vacuum at ambient to give the title compound (2.18 g, 99% th).

MPt 290° C. (decomposition).

¹H NMR 5(CD₃OD) 7.27 (1H, d, J=10 Hz), 6.24 (2H, dd, J=10 Hz), 6.19 (1H, s), 5.49 (1H, ddd), 4.14(1H, m), 2.45-2.60, (1H, m), 2.30-2.40 (3H, m), 2.24 (q, 2H, J=7 Hz), 2.05-2.15 (1H, m), 1.80-1.95(1H, m), 1.60 (3H, s), 1.15 (s, 3H), 1.05 (t, 3H, J=7 Hz), 0.88(d, 3H).

### 6α,9α-Difluoro-17α-(1-oxopropoxy)-11β-hydroxy-16α-methyl-3-oxo-androsta-1,4-diene-17β-carbothioic Acid Diisopropylethylamine Salt

A solution of 6α,9α-difluoro-17α-(1-oxopropoxy)-11β-hydroxy-16α-methyl-3-oxo-androsta-1,4-diene-17β-carbothioic acid (15 g) in acetone (80 ml) is stirred at ambient temperature for approximately 10 minutes under nitrogen blanketing. The mixture is cooled to 15° C. and diisopropylethylamine (5.73 ml) in acetone (20 ml) is added. The reaction mixture is aged for 0.5 hr at 20° C. and hexane (50 ml) is added over 15 minutes before cooling mixture to 15° C. and aging for a further 0.5 hr. The product is filtered off, washed with a chilled mixture of hexane (120 ml) and ethyl acetate (30 ml) and dried under vacuum at ambient temperature to give the title compound (17.56 g, 92%).

Mpt 182.1° C.

¹H NMR 8(DMSOd₆) 7.24 (1H, d), 6.28 (1H, d), 6.08 (1H, s), 5.63 (1H, ddd), 5.4 (1H, s) 5.25 (1H, bs), 4.06 (1H, m), 3.62 (2H, m), 3.25 (2H, m), 2.40 (2H, q), 2.15 (1H, m), 1.85-2.00 (2H, m), 1.45-1.6 (5H, s+m overlapped), 1.20-1.35 (17H, m), 1.15 (1H, s), 0.95-1.00 (1H, m), 0.90 (3H, s) and 0.66 (3H, d).

### 6α,9α-Difluoro-17α-(1-oxopropoxy)-11β-hydroxy-16α-methyl-3-oxo-androsta-1,4-diene-17β-carbothioic Acid 2,6-dimethylpyridine Salt

A solution of 6α,9α-difluoro-17α-(1-oxopropoxy)-11β-hydroxy-16α-methyl-3-oxo-androsta-1,4-diene-17β-carbothioic acid (2 g) in dichloromethane (20 ml) is stirred at approximately 22° C. for approximately 10 minutes under nitrogen. 2,6-Dimethylpyridine (0.46 g) in CH₂Cl₂ (10 ml) was added dropwise and stirred for 1 hr. Hexane (10 ml) is added dropwise and the mixture aged at 15° C. for at least 0.5 hr. This precipitated salt was filtered and washed with a chilled mixture of CH₂Cl₂ (10 ml) and hexane (30 ml) and dried under vacuum at ambient temperature to give the title compound (1.56 g, 63%).

Mpt 111° C.

¹H NMR 6(CDCl₃) 7.57 (1H, dd), 7.16 (1H, s), 7.04 (1H, d), 6.45 (1H, s), 6.42 (1H, dd), 5.40 (1H, ddd), 4.44 (1H, m), 3.34 (1H, bs), 2.60 (6H, s), 2.40 (4H, m), 2.20-2.35 (2H, m), 1.65-1.95 (3H, m), 1.55 (3H, s), 1.33 (1H, m), 1.10-1.20 (6H, m), 1.00 (3H, d).

### Example 8

### 6α,9α-Difluoro-17α-(1-oxopropoxy)-11β-hydroxy-16α-methyl-3-oxo-androsta-1,4-diene-17β-yl S-(1-oxopropoxy) thioanhydride

A solution of 6α,9α-difluoro-11β,17α-dihydroxy-16α-methyl-3-oxo-androsta-1,4-diene-17β-carbothioic acid (10 g) in acetone (125 ml) is cooled to approximately −5° C. and treated at 0 to −5° C. with triethylamine (16 ml) over approximately 15 minutes. The suspension is treated with propionyl chloride (8.5 ml) over approximately 90 minutes, maintaining the temperature at −5° C. to 0° C. and the solution is stirred at −5° C. to 0° C. for a further 2 hours. The reaction mixture is poured into 2M hydrochloric acid (470 ml) over ten minutes and the resultant suspension is aged at 5° C. for 30 minutes. The product is filtered off, washed with water (3×125 ml) and dried under vacuum at approximately 40° C. for 15 hours to give the title compound as a white to off white solid (12.78 g, 100.6%.

HPLC Retention Time 40.7 mins (99.5 Area % Purity)

CHN: Found C, 61.8%; H, 6.7%; S, 6.1%; C₂₇H₃₄F₂O₆S requires C, 61.8%; H, 6.5%; S, 5.7%.

NMR (DMSOd₆) 7.27 (1H, d, 10 Hz), 6.32 (1H, d, 10 Hz), 6.12 (1H, s), 5.81 (1H, d), 5.65 (1H, ddd), 4.37 (1H, m), 2.40 (2H, q, 7 Hz), 2.00-2.45 (4H, m), 1.85 (1H, m), 1.87 (1H, m), 1.51 (3H, s), 1.27 (1H, m), 1.11-1.22 (4H, m), 0.90-1.05 (6H, m), 0.88 (3H, d, 7 Hz).

### Example 9

### Preparation of 6α,9α-difluoro-11β,17α-dihydroxy-16α-methyl-3-oxo-androsta-1,4-diene-17β-carbothioic Acid Imidazole Salt (1:2)

6α,9α-Difluoro-11β-hydroxy-16α-methyl-3-oxo-17α-hydroxy-androsta-1,4-diene-17β-carboxylic acid (35 g, 0.089 moles) and carbonyldiimidazole (25.75 g, 0.16 moles) are stirred in ethyl acetate (350 ml) and N,N-dimethylformamide (17.5 ml) at 20±2° C. for 60 minutes. The suspension is cooled to 0° C. and the batch stirred at 0±5° C. whilst hydrogen sulphide (7.7 g, 0.23 moles) is added, via a sintered glass dip pipe, over 32 minutes. The batch is stirred at 0±3° C. for 30 minutes, warmed to 9° C. over 20 minutes and stirred at 9±3° C. for a total of 100 minutes. The product is collected by filtration (Whatman 54 paper) and the cake washed with ethyl acetate (2×105 ml). The product is dried under vacuum at approximately 20° C. for 20 hours to give the title compound as a white to pale purple solid (47.7 g, 98.5% th). NMR (MeOH_{d4}) 0.86 (3H) d, J=7.4 Hz; 1.11 (3H) s; 1.20 (1H) m; 1.61 (3H) s; 1.62-1.82 (3H) m; 2.14-2.25 (2H) m; 2.33 (1H) m; 2.54 (1H) m; 3.19 (1H) m; 4.26 (1H) ddd, J=11.2, 4.0, 1.8 Hz; 5.57 (1H) dddd, J=49.0, 11.6, 6.8, 1.9 Hz; 6.32 (1H) m; 6.35 (1H) dd, J=10.0, 2.0 Hz; 7.35 (4H), d, J=1.0 Hz (imidazole); 7.41 (1H) dd, J=10.0, 1.4 Hz; 8.30 (2H) t, J=1.0 Hz (imidazole).

MP 120° C. (decomp.)

### Example 10

### Preparation of a solution of 6α,9α-difluoro-11β,17α-dihydroxy-16α-methyl-3-oxo-androsta-1,4-diene-17β-carbothioic acid from 6α,9α-difluoro-11β,17α-dihydroxy-16α-methyl-3-oxo-androsta-1,4-diene-17β-carbothioic Acid Imidazole Salt (1:2) Imidazole Salt (1:2)

6α,9α-Difluoro-11β,17α-dihydroxy-16α-methyl-3-oxo-androsta-1,4-diene-17β-carbothioic acid imidazole salt (47.7 g) is stirred in ethyl acetate (811 ml) and the suspension is cooled to 15±3° C. 2M (aqueous) hydrochloric acid (286 ml) is added and the mixture is stirred for approximately five minutes affording a clear biphasic mixture. The layers are separated and the organic solution of the free carbothioic acid is washed with further 2M (aqueous) hydrochloric acid (190 ml).

Alternative Procedure

6α,9α-Difluoro-11β,17α-dihydroxy-16α-methyl-3-oxo-androsta-1,4-diene-17β-carbothioic acid imidazole salt (47.7 g) is stirred in pentan-3-one (954 ml) and the suspension is cooled to 15±3° C. 2M (aqueous) hydrochloric acid (286 ml) is added and the mixture is stirred for approximately five minutes affording a clear biphasic mixture. The layers are separated and the organic solution of the free carbothioic acid is washed with water (190 ml).

In either case, solvent wet cakes of 6α,9α-difluoro-11β,17α-dihydroxy-16α-methyl-3-oxo-androsta-1,4-diene-17β-carbothioic acid imidazole salt (rather than dried solids) can be used as inputs to the above acidification procedures.

### Example 11

### 6α,9α-Difluoro-17α-(1-oxopropoxy)-11β-hydroxy-16α-methyl-3-oxo-androsta-1,4-diene-17β-carbothioic Acid

Ethyl acetate (100 ml, 10 vol) and DMF (5 ml, 0.5 vol) were added sequentially to an intimate mixture of 6α,9α-difluoro-17α-(1-oxopropoxy)-11β-hydroxy-16α-methyl-3-oxoandrosta-1,4-diene-17β-carboxylic acid (10.0 g) and N,N′-carbonyldiimidazole (6.3 g). The resulting suspension was stirred at 17±3° C. for 50 minutes to give afford a pale yellow solution. The solution was cooled to 10° C. and hydrogen sulfide (2.2 g) was bubbled through the solution over 25 minutes maintaining the contents at 12±2° C. The resulting suspension was stirred at 12±2° C. for a further 90 minutes and then filtered. The cake was washed with ethyl acetate (2×30 ml) and sucked dry. The solid was then suspended in 3-pentanone (200 ml) and washed with 2M hydrochloric acid (60 ml) and then water (60 ml). The resulting solution was cooled to 3° C. and tripropylamine (14.0 ml) was added over 2 minutes ensuring the reaction remained at 3±2° C. The solution was stirred at 3±2° C. and propionyl chloride (5.3 ml) was added over 5 minutes keeping the reaction at 3±2° C. The solution was then allowed to warm to 10° C. and stirred at 12±2° C. for 90 minutes. The solution was then cooled to 3° C. and 1-methylpiperazine (5.1 ml) was added keeping the reaction at 3±2° C. The solution was stirred at 3±2° C. for 20 minutes, warmed to 18±3° C. and then washed sequentially with 1 M HCl (60 ml) and water (60 ml). One half of the solution (100 ml) was then treated with 2,2,4-trimethylpentane (100 ml) over 20 minutes. The resulting suspension was stirred at 20±3° C. for at least 14 hours and then filtered. The cake was washed with 3-pentanone: 2,2,4-trimethylpentane (2×20 ml) and sucked dry. The solid was dried in a vacuum oven at 40° C. for 6 hours to give the title compound as a white solid (4.1 g, 69% th).

### Example 12

### 6α,9α-Difluoro-17α-(1-oxopropoxy)-11β-hydroxy-16α-methyl-3-oxo-androsta-1,4-diene-17β-carbothioic Acid Triethylamine Salt

Ethyl acetate (100 ml) and DMF (5 ml) were added sequentially to an intimate mixture of 6α,9α-difluoro-17α-(1-oxopropoxy)-11β-hydroxy-16α-methyl-3-oxo-androsta-1,4-diene-17β-carboxylic acid (10.0 g) and N,N′-carbonyldiimidazole (6.3 g). The resulting suspension was stirred at 18-20° C. for 50 minutes to give afford a pale yellow solution. The solution was cooled to 10° C. and hydrogen sulfide (2.2 g) was bubbled through the solution over 25 minutes maintaining the contents at 12±2° C. The resulting suspension was stirred at 12±2° C. for a further 90 minutes and then filtered. The cake was washed with ethyl acetate (2×30 ml) and sucked dry. The solid was then suspended in 3-pentanone (200 ml) and washed with 2M hydrochloric acid (60 ml) and then water (60 ml). The resulting solution was cooled to 3° C. and tripropylamine (14.0 ml) was added over 2 minutes ensuring reaction remained at 3±2° C. The solution was stirred at 3±2° C. and propionyl chloride (5.3 ml) was added over 5 minutes keeping reaction at 3±2° C. The solution was then allowed to 10° C. and stirred at 12±2° C. for 90 minutes. The solution was then cooled to 3° C. and 1-methylpiperazine (5.1 ml) was added keeping the reaction at 3±2° C. The solution was stirred at 3±2° C. for 20 minutes, warmed to 18±3° C. and then washed sequentially with 1M HCl (60 ml) and water (60 ml). One half of the solution (100 ml) was cooled to 3° C. and treated with triethylamine (2.1 ml). The solution was stirred at 3±2° C. for 10 minutes, warmed to 20° C. and then 2,2,4-trimethylpentane (100 ml) was added over 20 minutes. The resulting suspension was stirred at 20±3° C. for at least 14 hours and then filtered. The cake was washed with 3-pentanone: 2,2,4-trimethylpentane (1:3, 2×20 ml) and sucked dry. The solid was dried in a vacuum oven at 40° C. for 6 hours to give the title compound as a white solid (6.2 g, 89% th).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

1. A process for preparing a compound of formula (II) 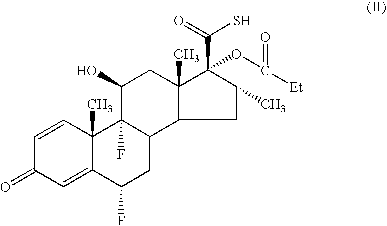
or a salt thereof
which comprises:
(a) reacting a compound of formula (III) 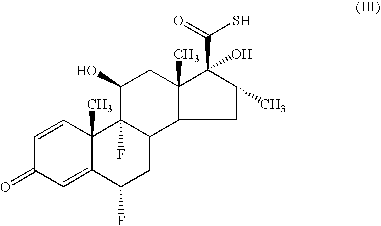
with an activated derivative of propionic acid in an amount of at least 1.3 moles of the activated derivative per mole of compound of formula (III) and;
(b) removal of the sulphur-linked propionyl moiety from any compound of formula (IIA) 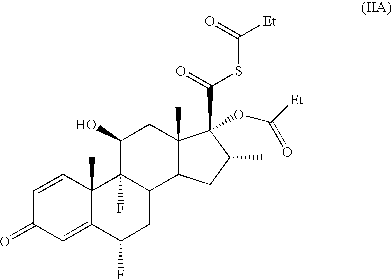
so formed by reaction of the product of step (a) with an organic primary or secondary amine base capable of forming a water soluble propanamide.

2. A process according to claim 1 wherein the organic base is diethanolamine or N-methylpiperazine.

3. A process according to claim 1 wherein the organic base is N-methylpiperazine.

4. A process according to claim 1 which further comprises the step of (c1), when the product of step (b) is dissolved in a substantially water immiscible organic solvent, purifying the compound of formula (II) by washing out the amide by-product from step (b) with an aqueous wash.

5. A process according to claim 1 which further comprises the step of (c2), when the product of step (b) is dissolved in a water miscible solvent, purifying the compound of formula (II) by treating the product of step (b) with an aqueous medium so as to precipitate out pure compound of formula (II) or a salt thereof.

6. A process according to claim 4 which comprises the further step of isolating the compound of formula (II) in the form of a solid crystalline salt.

7. A process according to claim 6 wherein the further step comprises treating the organic phase containing the compound of formula (II) with a counterion so as to precipitate the compound of formula (II) in the form of a solid crystalline salt.

8. A process according to claim 1 wherein the compound of formula (III) is used as its imidazole salt.

9. A process for preparing fluticasone propionate which comprises preparing a compound of formula (II) or a salt thereof according to claim 1 and converting the compound of formula (II) or salt thereof to fluticasone propionate by treatment with a compound of formula LCH₂F wherein L represents a leaving group.

10. A compound of formula (II) 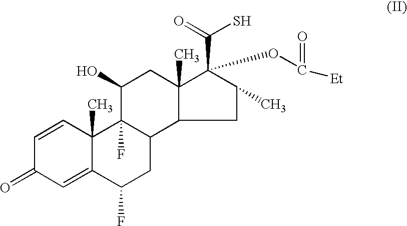
isolated in the form of a solid crystalline salt.

11. A compound of formula (II) according to claim 10 isolated as the triethylamine salt.

12. A compound of formula (II) according to claim 10 isolated as the potassium salt.

13. A compound of formula (IIA)

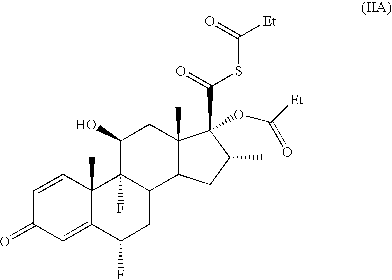

14. A compound which is an imidazole salt of the compound of formula (III) as defined in claim 1.

15. A process for the preparation of an imidazole salt of the compound of formula (III) as defined in claim 1 which process comprises the reaction of a compound of formula (IV) 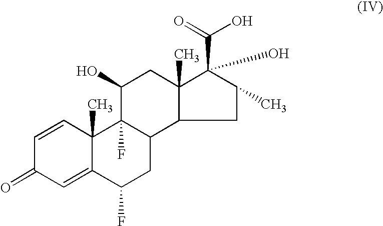
with carbonyldiimidazole and hydrogen sulphide.
